(19) United States
(12) Patent Application Publication
(10) Pub. No.: US 2008/0045805 A1
(43) Pub. Date: Feb. 21, 2008

(54) Method and System of Indicating a Condition of an Individual

(76) Inventors: Oded Sarel, Even Yehuda (IL); Yoram Levanon, Ramat Hasharon (IL); Lam Lossos, Zur Hadassa (IL)

Correspondence Address: GIFFORD, KRASS, SPRINKLE,ANDERSON & CITKOWSKI, P.C, PO BOX 7021, TROY, MI, 48007-7021, US

(21) Appl. No.: 11/720,442

(22) PCT Filed: Nov. 30, 2005
(86) PCT No.: PCT/IL05/01277

§ 371 Date: May 30, 2007

Related U.S. Application Data
(60) Provisional application No. 60/631,511, filed on Nov. 30, 2004.

(51) Int. Cl. A61B 5/00

(52) U.S. Cl. 600/300

(57) ABSTRACT

A system and method of indicating a condition of a tested individual, wherein sounds generated during testing the individual are processed to define a match with predefined criteria, wherein at least part of the received sounds are not discernible to human ears and at least some of the sounds are generated when the individual is mute.

### FIELD OF THE INVENTION

This invention relates to methods and system capable of indicating a condition of an individual and, in particular, hidden intent, emotion and/or thinking activity.

### BACKGROUND OF THE INVENTION

Systems for identification of an individual's condition by registration and analysis of changes in psycho-physiological characteristics in response to questions or other stimuli and interpretation of corresponding hidden intent, emotions and/or thinking activity, are known in the art. In addition to classical polygraph techniques of registering galvanic skin response, respiration rate, heart rate and blood pressure, the prior art includes also registering and analyzing of other changes in the body that cannot normally be detected by human observation. For example, known improvements of the classical polygraph use electro-encephalography to measure P3 brain-waves (e.g. U.S. Pat. No. 4,941,477 (Farwell), U.S. Pat. No. 5,137,027 (Rosenfeld) and later U.S. Pat. No. 6,754,524 (Johnson)); a pen incorporating a trembling sensor to ascertain likely signs of stress (U.S. Pat. No. 5,774,571 (Marshall)), a hydrophone fitted into a seat to measure voice stress levels, heart and breath rate, and body temperature (U.S. Pat. No. 5,853,005 (Scanlon)).

Some methods of detecting an individual's condition are based on voice and/or speech analysis. For example, U.S. Pat. No. 3,971,034 discloses a method of detecting psychological stress by evaluating manifestations of physiological change in the human voice wherein the utterances of a subject under examination are converted into electrical signals and processed to emphasize selected characteristics which have been found to change with psycho-physiological state changes. The processed signals are then displayed on a strip chart recorder for observation, comparison and analysis. Infrasonic modulations in the voice are considered to be stress indicators, independent of the linguistic content of the utterance.

U.S. Pat. No. 6,006,188 (Bogdashevsky et al.) discloses a speech-based system for assessing the psychological, physiological, or other characteristics of a test subject. The system includes a knowledge base that stores one or more speech models, where each speech model corresponds to a characteristic of a group of reference subjects. Signal processing circuitry, which may be implemented in hardware, software and/or firmware, compares the test speech parameters of a test subject with the speech models. In one embodiment, each speech model is represented by a statistical time-ordered series of frequency representations of the speech of the reference subjects. The speech model is independent of a priori knowledge of style parameters associated with the voice or speech. The system includes speech parameterization circuitry for generating the test parameters in response to the test subject's speech. This circuitry includes speech acquisition circuitry, which may be located remotely from the knowledge base. The system further includes output circuitry for outputting at least one indicator of a characteristic in response to the comparison performed by the signal processing circuitry. The characteristic may be time-varying, in which case the output circuitry outputs the characteristic in a time-varying manner. The output circuitry also may output a ranking of each output characteristic. In one embodiment, one or more characteristics may indicate the degree of sincerity of the test subject, where the degree of sincerity may vary with time. The system may also be employed to determine the effectiveness of treatment for a psychological or physiological disorder by comparing psychological or physiological characteristics, respectively, before and after treatment.

U.S. Pat. No. 6,427,137 (Petrushin) teaches a system, method and article of manufacture for a voice analysis system that detects nervousness for preventing fraud.

U.S. Pat. No. 6,591,238 (Silverman) discloses a method for electronically detecting human suicidal predisposition by analysis of an elicited series of vocal utterances from an emotionally disturbed or distraught person independent of linguistic content of the elicited vocal utterance.

US Patent Application No. 2004/0093218 (Bezar) shows a speaker intent analysis for validating the truthfulness and intent of a plurality of participants' responses to questions. The data processor analyzes and records the participants' speech parameters for determining the likelihood of dishonesty.

### SUMMARY OF THE INVENTION

As is well-known in the art, sounds are generated by air flow through the various components in the vocal tract. Humans can produce sounds in a frequency range of about 8-20,000 Hertz. Normal human hearing is able to detect a frequency range between approximately 60 and 16,000 Hertz. Thus, the vocal tract can generate sounds beyond the frequencies which the human ear can hear. Sounds with frequencies below 65 Hertz are called infrasonic and those higher than 16,000 Hertz are called ultrasonic.

Sound production by the vocal tract involves various muscular contractions; even small changes in muscular activity lead to frequency and amplitude changes in the sound output. In addition, the various vocal articulators, such as the tongue, soft palate, and jaw are connected to the larynx in various ways, and thus can affect vocal fold vibration. Fluctuations in sound output (volume, shape, etc.) may be caused by an influx of blood flow through the vocal tract elements as well as by other physiological reasons.

The inventors have found a correlation between an individual's condition (e.g. emotional arousal, thinking activity, etc.) and frequency and volume changes in ultrasonic and/or infrasonic sounds generated while speaking and/or while being mute. These changes can be measured and analyzed. While the ability to skillfully control the pressure and flow of air is a large part of successful voice use, individuals cannot control the generation of infrasonic and ultrasonic sounds.

The invention, in some of its aspects, is aimed to provide a novel solution capable of facilitating indication of an individual's condition (e.g. intents, emotions, thinking activity, etc.) The indication is based on registration of sounds generated by an individual a) when the individual is speaking and is mute during the registration; and/or b) when the individual is mute for the entire duration of the registration.

In accordance with certain aspects of the present invention, there is provided a method of indicating a condition of a tested individual, the method comprising:

receiving received sounds generated during testing the individual; and

processing at least some of the received sounds so as to define a match with predefined criteria;

wherein at least some of the received sounds are not discernible by the human ear and at least some of said received sounds are generated when the individual is mute.

In accordance with further aspects of the invention, there is provided a system for indicating a condition of a tested individual in accordance with the invention includes a receiving unit for registering sounds generated during testing the individual and a processor coupled to the registration unit for processing at least some of said received sounds to define a match with predefined criteria, wherein at least some of the received sounds are not discernible to the human ear and at least some of said received sounds are generated when the individual is mute.

The processor may be coupled directly to registration unit or may be coupled remotely thereto so that the processing may be done independent of the actual sound registration.

### BRIEF DESCRIPTION OF THE DRAWINGS

In order to understand the invention and to see how it may be carried out in practice, an embodiment will now be described, by way of non-limiting examples only, with reference to the accompanying drawings, in which:

FIG. 1 illustrates a generalized block diagram of exemplary system architecture, in accordance with an embodiment of the invention;

FIG. 2 illustrates a generalized flow diagram showing the principal operations for operating the test in accordance with an embodiment of the invention; and

FIG. 3 illustrates a generalized flow diagram showing the principal operations of a decision-making algorithm in accordance with an embodiment of the invention.

### DETAILED DESCRIPTION OF EXEMPLARY EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention. In the drawings and descriptions, identical reference numerals indicate those components that are common to different embodiments or configurations.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or processor or similar electronic computing device, that manipulate and/or transform data represented as physical, such as electronic, quantities within the computing system's registers and/or memories into other data, similarly represented as physical quantities within the computing system's memories, registers or other such information storage, transmission or display devices.

Throughout the following description the term “memory” will be used for any storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions that are capable of being conveyed via a computer system bus. The term “database” will be used for a collection of information that has been systematically organized, typically, for an electronic access.

The processes/devices presented herein are not inherently related to any particular electronic component or other apparatus, unless specifically stated otherwise. Various general purpose components may be used in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

Above-referenced prior art applications teach many principles of converting voice into electrical signals and assessing characteristics of an individual's condition. Therefore the full contents of these publications are incorporated herein by reference.

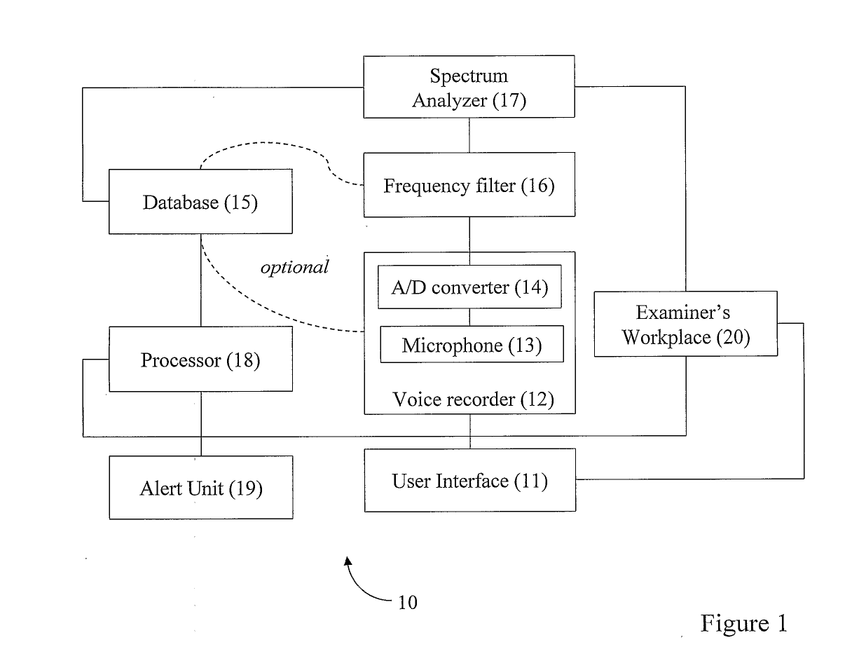

Referring to FIG. 1, there is schematically illustrated a system 10 for indicating an individual's conditions (e.g. hidden intents, emotions, thinking activities, etc.) in accordance with an embodiment of the invention.

A user interface 11 is connected to a voice recorder 12. The user interface contains means necessary for receiving sounds from the individual and for providing stimuli (e.g. questions, images, sounds, etc.). The sounds may be received directly from the individual as well as remotely, e.g. via a telecommunication network. The user interface 11 may comprise a workstation equipped with one or more microphones able to receive sounds in ultrasonic and/or infrasonic frequency bands and transmit the sounds and/or derivatives thereof. The user interface 11 may also comprise different tools facilitating exposure of stimuli to the individual being tested, e.g. display, loudspeaker, sound player, stimuli database (e.g. questionnaires), etc. The user interface 11 transmits the received sounds (e.g. voice including infrasonic and/or ultrasonic bands or just sounds generated by the tested individual during muting and not discernible to human ears) to a voice recorder 12 via direct or remote connection.

The voice recorder 12 is responsive to a microphone 13 capable of receiving and recording sounds and/or derivatives thereof at least in the ultrasonic and/or infrasonic frequency bands. An analog-digital (A/D) converter 14 is coupled to the microphone 13 for converting the sounds received from an individual into digital form. The recorded sounds may be saved in a database 15 in analog and/or digital forms. Connection of the voice recorder to the database is optional and may be useful for storing of sound records for, e.g., forensic purposes or for optimization of the analysis process. The A/D converter 14 is connected to at least one frequency filter 16 capable of filtering at least ultrasonic and/or infrasonic bands and/or sub-bands thereof. The frequency filter 16 filters predefined bands and/or sub-bands and transmits them to a spectrum analyzer 17 for detecting volumes at various frequencies.

Although the microphone 13 is shown as part of the voice recorder 12 it may be a separate unit connected thereto. Likewise, the A/D converter 14 may be a separate unit connected to the voice recorder, or it may be integrated with the microphone 13 as a separate unit, or it may be provided by a telecommunication network between the microphone 13 and the voice recorder 12 or between the voice recorder 12 and the frequency filter 16.

In certain embodiments of the present invention the predefined sub-bands may be one or more octaves apart, such that the ratios of the corresponding frequencies in different sub-bands are multiples of two.

In certain embodiments of the invention the predefined bands/sub-bands may be selected from the following group wherein the selection comprises at least a) or b) categories:

a) bands/sub-bands comprising discernible and non-discernible frequencies within one band/sub-band;

b) bands/sub-bands comprising only non-discernible frequencies within one band/sub-band;

c) bands/sub-bands comprising bands/sub-bands comprising only discernible frequencies within one band/sub-band.

A spectrum analyzer 17 is connected to the database 15. Optionally, the database 15 may be connected to the frequency filter(s) for storing the filtered records.

The database 15 stores results obtained by the spectrum analyzer 17 and, optionally, the entire sound records and/or ultrasonic and infrasonic parts of the records. The individual's sound records may be obtained and analyzed also before the test and/or during an initial part of the test. The database 15 may also contain sound records and/or derivatives thereof obtained from different people placed under the same conditions as the individual being tested (e.g., the same neutral situation, the same stimuli, their consequence, etc.). The mixture of records and/or derivatives thereof can be used for creating a baseline for further comparison with results of the individual's response to the stimuli. The database 15 may store baselines previously calculated for different test scenarios.

A variety of test scenarios may be stored either in the database 15 or in association with the user interface 11. The database 15 may also contain data about stimuli and test scenarios implemented during the individual's testing. In certain embodiments of the invention the database 15 may store substantially all sound records obtained during the individual's testing; later, in accordance with a test scenario, some of these records may be used for creating an individual's sound pattern while others, synchronized with the stimuli, may be used for analysis of appropriate changes in ultrasonic and/or infrasonic frequency bands.

The database 15 may also contain data relating to evaluation procedures, including test criteria and predefined discrepancies for different test scenarios as well as rules and algorithms for evaluation of any discrepancy between registered parameters and test criteria. Test criteria may be Boolean or quantified, and may refer to a specific record and/or group of records and/or derivatives thereof. Discrepancies may be evaluated against the baseline and/or on the individual's personal pattern.

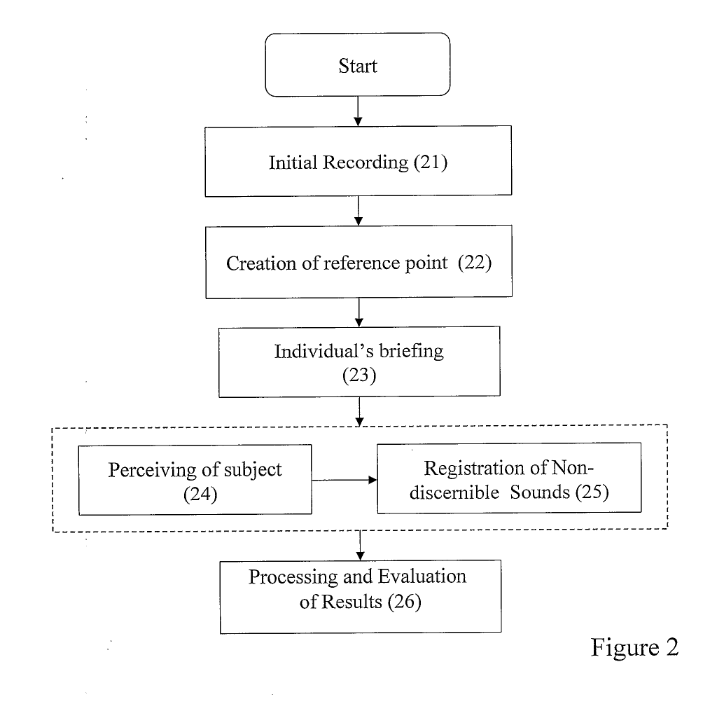

A processor 18 is connected to the database 15 and for processing the stored data. It may also provide management of data stored in the database 15 as well as management of test scenarios stored in the database 15 and/or the user interface 11. The processor executes calculations and data management necessary for evaluation of results obtained by the spectrum analyzer 17 and to determine a discrepancy in the individual's response to the exposed stimuli. The processor may contain algorithms and programs for analysis of the spectrum and evaluation of obtained results. Optionally, if the detected discrepancy corresponds to a predefined malicious range, the processor 18 will send a notice to an alert unit 19, providing, e.g. audio, visual or telecommunication (e.g. SMS or e-mail) indication. As will be further detailed with reference to FIG. 2, the processor, in accordance with the implemented test scenario, selects an appropriate baseline, individual's personal pattern or other test criteria. In certain embodiments of the invention the processor may, if necessary, calculate a new baseline and/or pattern for the purpose of the test. The above processing functionality may be distributed between various processing components connected directly or indirectly.

The system 10 further includes an examiner's workplace 20 that facilitates the test's observation, management and control and may, to this end, include a workstation or terminal with display and keyboard that are directly or indirectly connected with all components in the system 10. In other embodiments the examiner's workplace 20 may be connected to only some components of the system, while other components (e.g. spectral analyzer) may have built-in management tools and display or need no management tools (e.g. A/D converter).

Those skilled in the art will readily appreciate that the invention is not bound by the configuration of FIG. 1; equivalent functionality may be consolidated or divided in another manner. In different embodiments of the invention, connection between the blocks and within the blocks may be implemented directly or remotely. The connection may be provided via Wire-line, Wireless, cable, Voice over IP, Internet, Intranet, or other networks, using any communications standard, system and/or protocol and variants of evolution thereof.

The functions of the described blocks may be provided on a logical level, while being implemented (or integrated with) different equipment. The invention may be implemented as an integrated or partly integrated block within testing or other equipment as well as in a stand-alone form. The assessing of individual's conditions based on non-discernible sounds registered in accordance with the present invention, may be provided by different methods known in the art and evolution thereof.

Referring to FIG. 2 there is schematically illustrated the principal operations for performing a test in accordance with an exemplary embodiment of the invention.

The invention provides embodiments for “cooperative” and “non-cooperative” procedures. In the case of “cooperative” procedures, a tested individual collaborates (or partly collaborates) with an examiner during the test, e.g. during a polygraph investigation, examination by psychologist or doctor, etc. The “non-cooperative” procedure supposes a lack of cooperation between the examiner and the tested individual. In some embodiments, the testing may be provided with no awareness by the individual of being under control.

In some embodiments, the invention may be implemented for different purposes including, but not limited to:
as an enhancement or separate system in the field of security, e.g., for polygraph, systems for border control, voice recognition, etc;
as an assistant tool for medical examinations, e.g. during detecting or diagnosing of certain diseases such as psychiatric disorders, etc.
as an assistant tool for study during therapy, e.g. while inspecting the effectiveness of sedative medicine;
as a tool for psychology investigations, e.g. to monitor reactions to words, matters, names, etc. during a treatment; estimation of personal affiliation with different subjects, etc.
as a truthfulness or stress analyzer for business purposes, trustworthiness tests of human resources in sensitive organizations;
as a tool for bio-feedback training;

As illustrated in FIG. 2, at the beginning of the process during initial period (21) the systems starts recording sounds generated by the individual being tested. The recorded sounds may be continuous or contain several samples (e.g. recorded in several tens of seconds).

In certain embodiments of the invention the initial period may be neutral, while in other embodiments it may comprise stimuli enabling investigating some pre-defined conditions of the individual. In certain embodiments of the invention the individual may be mute during the initial period, while in other embodiments he/she may speak during at least part of this period. In certain embodiments of the invention the registration may be provided with respect to non-discernible sounds, while in other embodiments the registration may be provided with respect to both non-discernible and discernible sounds.

The recorded spectra are analyzed and processed by the system to provide at least one reference for further analysis. In accordance with certain embodiments of the invention, the reference may be a personal pattern created as a result of processing the recorded sounds generated by the tested individual during the initial period. The reference may be also a baseline created, for example, as a result of processing the recorded sounds generated by different individuals during testing under appropriate conditions, as a result of theoretical calculations, as a result of a prior knowledge in appropriate areas, etc. The appropriate baseline may be selected in accordance with data recorded during the initial period or, for example, in accordance with the nature of the tests, individual's personal information, etc.

In the embodiment illustrated by way of non-limiting example in FIG. 2, the reference is a personal pattern that is created (22) based on sounds recorded during the initial period. In certain embodiments of the invention the personal pattern may be created in reasonable time before start of the tests.

The next stage in a “cooperative” embodiment of the invention is individual's briefing (23) of subjects (e.g. matters, terms, names, etc.) intended for following investigations. At the next stage (24) the individual concentrates on the above subjects. The perceiving process may involve thinking about the matters, process of utterance, mute pronouncing of the words with closed mouth and/or with articulation, etc. In certain embodiments of the invention the pronouncing may be mute and/or with voice. For each of the investigating subjects, the system records (25) sound non-discernible by the human ear in parallel with perceiving the selected matter by the individual. The process is repeated for each subject being investigated. In certain embodiments of the invention the system may record non-discernible sounds when the individual is mute, while in other embodiments the system may record non-discernible sounds or both non-discernible and discernible sounds when the individual is speaking and/or is mute.

The analysis of the recorded sounds (including analysis desired for pattern creation) may include calculation of minimal, average and/or maximal volumes in recorded bands/sub-bands (e.g. sub-bands around 30, 35 and 40 Hz and/or sub-bands around 12, 17 and 20 KHz). In certain embodiments of the invention the calculations may comprise a signal amplitude decay, degree or amount of amplitude modulation or any other calculation suitable for testing an individual's condition and known in the art. In certain embodiments of the invention the recorded (and/or analyzed) sub-bands may be one or more octaves apart and the calculations may compare the volumes (or other parameters) at frequencies with ratios as multiples of two, and the analysis may comprise any repetitive changes at such frequencies.

In accordance with further aspects of the present invention, the inventors have found a correlation between thinking activity (e.g. emotional and non-emotional thought, internal speech, etc.) and repetitive changes (e.g. decays or peaks) at frequencies being substantially one octave apart within sub-bands 16-32 Hertz and 32-64 Hertz. In accordance with certain embodiments of the present invention, assessing the thinking activity (regardless of emotions, stress, etc.) comprises analysis of at least records made in sub-band 16-32 Hertz and in sub-band 32-64 Hertz staying an octave apart. The analysis may comprise comparing repetitive changes (e.g. decays or peaks) at frequencies being one octave apart as well as volume and other parameter changes in the specified sub-bands.

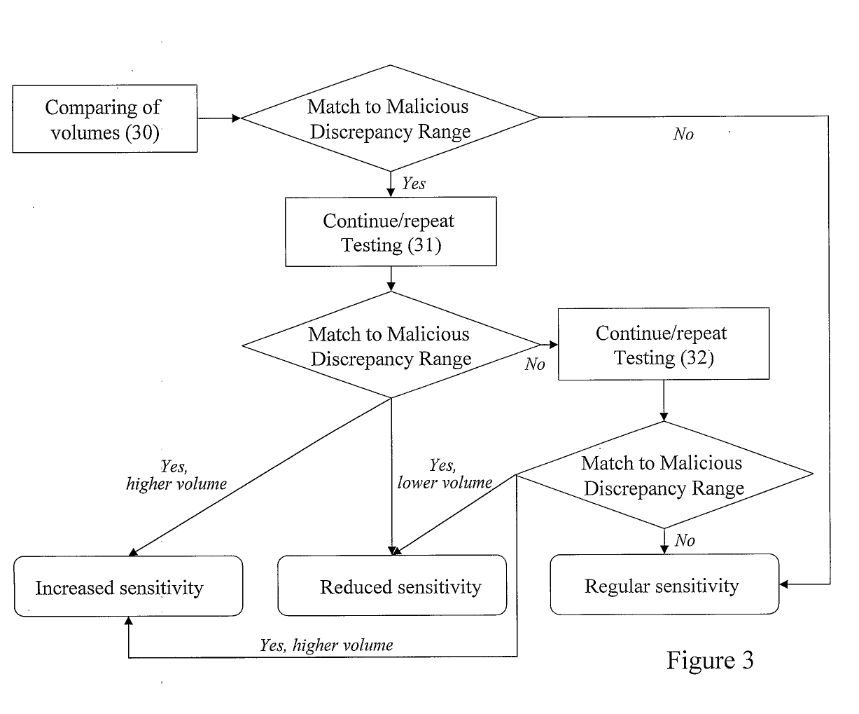

The processing and evaluation of results (26) includes discrepancy evaluation which comprises comparing the recorded sounds and/or derivatives thereof (e.g., results of spectral analysis for each of investigated matters) with test criteria in accordance with pre-defined rules and algorithms for evaluation. The recorded spectra may be analyzed and processed in a manner similar to the pattern creation (22). Test criteria may be defined as the individual's personal pattern, selected baseline and/or derivatives thereof. The evaluated discrepancy (if any) is compared with the pre-defined malicious discrepancy range as further detailed with reference to FIG. 3. A discrepancy matching the pre-defined malicious discrepancy range may cause any type of alert, depending on a specific embodiment of the invention. The degree of discrepancy may serve as an indication of, for example, sensitivity level as, by way of non-limiting example, further illustrated with reference to FIG. 3.

Those versed in the art will readily appreciate that the invention is not bound by the sequence of operations illustrated in FIG. 2.

The following test illustrates, by way of non-limiting example, a “cooperative” embodiment of the present invention. The test was provided for estimating a level of emotional reaction of twenty five volunteers who were asked to scale an importance of four different terms (e.g. mother, father, health, money) on a 1 to 10 scale, and keep the report. Later the volunteers were asked to think about each of the terms separately, and resulting non-discernible sounds were registered and analyzed in accordance with the method illustrated with reference to FIGS. 2 and 3. The resulting estimations of emotional reaction were compared with the kept records as summarized in Table 1.

TABLE 1

| Result | Number of terms |
|---|---|
| No difference with kept report by volunteer | 80 |
| 1 degree difference (e.g. scale of 7 by test | 16 |
| instead of 8 in the kept report) | |
| 2 degree difference (e.g. scale of 4 by test | 1 |
| instead of 6 in the kept report) | |
| 3 degree difference (e.g. scale of 7 by test | 3 |
| instead of 10 in the kept report) | |
| Total: | 100 |

A short dialog on a border control may illustrate a non-cooperative embodiment of the invention. In accordance with certain embodiments of the present invention, such dialog may provide indications of stress while the individual does not know that he is under control. Examples of such short dialogs include:
“Where have you come from? What is your flight number?”—during this neutral part of the dialog the system creates an individual's pattern by registering infra and/or ultra-sonic voice bands when the individual is speaking, as well as non-discernible sounds while the individual is mute.
“May I check your luggage, please? Please open and switch on your laptop. What is the purpose of your visit? etc.”—the system analyses sound records during such questions which have the potential to arouse emotions, and compares them with the created individual's pattern to establish whether there is a discrepancy and, if discovered, whether it is suggestive of hidden emotions or intent related to the questions.

Control of an individual's sensitivity and/or attitude during medical or psychological treatment may be implemented in a similar manner. An examiner may create an individual's pattern based on response to neutral and sensitive words and questions. Such a pattern will allow the examiner to identify sensitive matters and words, recognize them while a patient is speaking or is mute and follow-up changes (if any) of sensitivity during the course of treatment.

The discrepancy evaluated in accordance with certain embodiments of the present invention may be used for bio-feedback training wherein the individual can monitor the discrepancy between the current response and a desired reference and, thus, consciously control his/her condition (e.g. emotions, concentration, reaction, etc.).

In a similar manner the present invention may be implemented, for example, for indicating thinking activity during cognitive tests. For example, during an initial period, the tested individual is asked to perform some simple arithmetical operations in order to create a “thinking” personal pattern as described with reference to FIGS. 1 and 2 (e.g. in a sub-band around 40 Hertz). The discrepancy against this pattern will provide indication of increased or decreased thinking activity.

The attention is now drawn to FIG. 3 schematically illustrating a flow diagram showing the principal operations of a decision-making algorithm in accordance with an embodiment of the invention.

In the illustrated embodiment, by way of non-limiting example, the evaluation of sensitivity for a specified matter or word is based on comparing (30) minimal, average and/or maximum volumes in each selected sub-band of the individual's characteristic pattern with the volumes of respective frequencies recorded during the individual's perceiving of the investigated matter/word. If the discrepancy does not match the pre-defined malicious discrepancy range, the system will consider the sensitivity to the matter as regular. If the discrepancy matches the malicious range, the test will be repeated (31). If the new discrepancy matches the malicious range, the system will provide an indication of increased sensitivity for the tested matter if the discrepancy is positive; and of reduced sensitivity if the discrepancy is negative. If, in contrast to results of the comparing operation (30), the new discrepancy does not match the pre-defined malicious discrepancy range, the test is repeated (32) and interpreted in the above manner.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present invention.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

Those skilled in the art will readily appreciate that various modifications and changes can be applied to the embodiments of the invention as hereinbefore described without departing from its scope, defined in and by the appended claims.

1-53. (canceled)

54. A method of indicating a condition of a tested individual, the method comprising: receiving sounds generated during testing the individual; and processing some or all of the received sounds within a predefined frequency range, so as to define a match with predefined criteria; wherein some or all of the received sounds are not discernible by the human ear and at least some of said received sounds are generated when the individual is either mute or speaking.

55. The method in accordance with claim 54, wherein the individual is mute or during inter-word silences.

56. The method in accordance with claim 54, wherein the individual is speaking.

57. The method in accordance with claim 54, wherein the predefined frequency range comprises two or more sub-bands; possibly wherein each frequency component in the second sub-band has a frequency that is a multiple of a corresponding frequency component in the first sub-band; and

58. The method in accordance with claim 57 wherein said range comprises at least two sub-band being substantially one octave apart from another sub-band.

59. The method in accordance with claim 54, wherein the processing includes comparing a respective response of corresponding frequency components that have frequency ratios substantially equal to multiples of two.

60. The method of claim 55, where the first and second sub-bands have frequencies that lie substantially within a range of 16-32 Hertz and 32-64 Hertz, respectively and the processing comprises comparing a respective response of corresponding frequency components that have frequency ratios substantially equal to multiples of two.

61. The method in accordance with claim 54, wherein the non-discernible sounds are selected from the group consisting of infrasonic, ultrasonic and a combination thereof.

62. The method in accordance with claim 54, wherein the predefined criteria is one or more of the group of (i) a personalized pattern of the individual and the condition of the tested individual is characterized by a discrepancy in matching the pattern to test data; and, (ii) baseline and the condition of the tested individual is characterized by a discrepancy between the baseline and test data.

63. The method in accordance with claim 62 wherein said at least one criteria is selected from the group consisting of (i) hidden intent, (ii) emotion, and (iii) thinking activity.

64. The method in accordance with claim 54, being performed in response to an indication selected from the group consisting of: the individual knowing of the testing, individual unknowing of the testing, polygraph testing, border control testing, voice recognition, inspecting effectiveness of a medicine or treatment, psychological investigation, testing trustworthiness, analyzing stress, and a combination thereof.

65. A system for indicating a condition of a tested individual, the system comprising a receiving unit for receiving sounds within a predefined frequency range generated during testing the individual and a processor coupled to the receiving unit for processing all or some of said received sounds to define a match with predefined criteria, wherein all or some of the received sounds are not discernible to the human ear and all or some of said received sounds are generated when the individual is either mute or speaking.

66. The system in accordance with claim 65, wherein the individual is mute or during inter-word silences.

65. The method in accordance with claim 65, wherein the individual is speaking.

66. The system in accordance with claim 65, wherein the predefined frequency range comprises one or more sub-bands.

67. The system in accordance with claim 66 wherein said range comprises at least one sub-band being substantially one octave apart from another sub-band.

68. The system in accordance with claim 65, including a sound processing device having corresponding frequency ratios substantially equal to multiples of two.

69. The system in accordance with claim 65, adapted to process sounds with frequencies substantially within a sub-band 16-32 Hertz and a sub-band 32-64 Hertz.

70. The system in accordance with claim 65, wherein the non-discernible sounds are selected from the group consisting of infrasonic, ultrasonic or combination thereof.

71. The system in accordance with claim 65, wherein the predefined criteria is one or more of the group of (i) a personalized pattern of the individual and the condition of the tested individual is characterized by a discrepancy in matching the pattern to test data; and, (ii) baseline and the condition of the tested individual is characterized by a discrepancy between the baseline and test data.

72. A computer program comprising computer program code operating on a computer for performing the method of claim 54.

73. A computer program as claimed in claim 72, embodied on a computer readable medium.
